(19) United States
(12) Patent Application Publication
(10) Pub. No.: US 2021/0382070 A1
(43) Pub. Date: Dec. 9, 2021

(54) DIAGNOSTIC BIOMARKERS FOR SHRIMP ALLERGY

(71) Applicant: The Chinese University of Hong Kong, Shatin (CN)

(72) Inventors: Ting Fan LEUNG, Shatin (CN); Yee Yan Christine Wai, Kowloon (CN); Sze Yin Agnes Leung, North Point (CN); Yat Hin Nicki Leung, Kowloon (CN)

(21) Appl. No.: 17/335,602

(22) Filed: Jun. 1, 2021

Related U.S. Application Data
(60) Provisional application No. 63/034,893, filed on Jun. 4, 2020.

(51) Int. Cl. G01N 33/68; A61K 49/00; G01N 33/50

(52) U.S. Cl. CPC G01N 33/6893; G01N 2800/24; G01N 33/505; A61K 49/0006

(57) ABSTRACT

The present invention provides methods and compositions for improved diagnosis of shrimp allergies.

### RELATED APPLICATIONS

This application claims priority to U.S. Provisional Patent Application No. 63,034,893, filed Jun. 4, 2020, the contents of which are hereby incorporated by reference in the entirety for all purposes.

### BACKGROUND OF THE INVENTION

Current diagnosis of shrimp allergy relies on a skin prick test (SPT) and a measurement of specific IgEs to commercial shrimp extracts. However, these methods are far from satisfactory, with high false positive rates due to the presence of cross-reactive allergens and variable allergen contents in the commercial shrimp extracts. They also only reflect sensitization but not clinical allergy. Although oral food challenge represents the gold standard for food allergy diagnosis, it is risky and labor-intensive, which hamper its clinical implementation. Component testing using tropomyosin, which has long been identified as the major shrimp allergen, has been shown to improve diagnostic accuracy, but this allergen represents only a minor allergen in Asia-Pacific populations, including in Hong Kong, and therefore has low diagnostic value in this context.

There is therefore an unmet need for new and more reliable diagnostic markers relevant to Asia-Pacific populations for the accurate diagnosis of shrimp allergy. The present invention satisfies this need and provides other advantages as well.

### BRIEF SUMMARY OF THE INVENTION

Numerous embodiments of the present invention, including compositions and methods for their preparation and administration, are presented herein.

In one aspect, the present disclosure provides a method for detecting a shrimp allergy in a subject, the method comprising: obtaining a serum or plasma sample from the subject; contacting the serum or plasma sample with shrimp Troponin C (TnC) and/or Fatty acid-binding protein (FABP); and detecting the level of IgE binding to the shrimp TnC and/or FABP in the serum or plasma sample; wherein a detection of a greater amount of IgE binding in the serum or plasma sample to shrimp TnC and/or FABP as compared to the amount in serum or plasma from a non-allergic control indicates that that subject has a shrimp allergy.

In another aspect, the present disclosure provides a method for detecting a shrimp allergy in a subject, the method comprising: contacting a serum or plasma sample from the subject with shrimp TnC and/or FABP; and detecting the level of IgE binding to the shrimp TnC and/or FABP in the serum or plasma sample; wherein a detection of a greater amount of IgE binding in the serum or plasma sample to shrimp TnC or FABP as compared to the amount in serum or plasma from a non-allergic control indicates that that subject has a shrimp allergy.

In some embodiments of either of the above disclosed methods, the IgE-binding assay is an ELISA or ImmunoCAP assay. In some embodiments, the shrimp TnC and/or FABP is recombinant and/or derived from Penaeus monodon.

In another aspect, the present disclosure provides a method for detecting a shrimp allergy in a subject, the method comprising: obtaining a blood sample from the subject; contacting the blood sample with shrimp TnC and/or FABP; and detecting the level of IgE crosslinking in immune cells in the blood sample in the presence of the shrimp TnC and/or FABP; wherein a detection of a greater amount of IgE crosslinking in immune cells in the blood sample in the presence of the TnC and/or FABP as compared to the amount in blood from a non-allergic control indicates that that subject has a shrimp allergy.

In another aspect, the present disclosure provides a method for detecting a shrimp allergy in a subject, the method comprising: contacting a blood sample from the subject with shrimp TnC and/or FABP; and detecting the level of IgE crosslinking in immune cells in the blood sample in the presence of the shrimp TnC and/or FABP; wherein a detection of a greater amount of IgE crosslinking in immune cells in the blood sample in the presence of the shrimp TnC and/or FABP as compared to the amount in blood from a non-allergic control indicates that that subject has a shrimp allergy.

In some embodiments of either of the above-disclosed methods, the IgE crosslinking assay is a basophil activation test (BAT). In some embodiments, the BAT is performed using TnC and/or FABP. In some embodiments, basophil activation is detected by detecting increased numbers of CD63⁺ basophils in the blood sample. In some embodiments, the shrimp TnC and/or FABP is recombinant and/or derived from Penaeus monodon.

In another aspect, the present disclosure provides a method for detecting a shrimp allergy in a subject, the method comprising: applying an amount of a solution comprising shrimp TnC and/or FABP to the skin of the subject; pricking the skin of the subject at the location of the applied solution; and assessing the reaction of the skin at the location of the skin prick after a specified amount of time; wherein an allergic reaction of the skin at the site of the skin prick indicates that the subject has a shrimp allergy.

In some embodiments of the method, the TnC and/or FABP are recombinant and/or derived from Penaeus monodon. In some embodiments, the specified amount of time is 15 minutes. In some embodiments, the allergic reaction of the skin comprises a wheal of at least 3 mm in diameter. In some embodiments, the skin prick is performed on the volar surface of the arm.

In another aspect, the present disclosure provides a method for detecting a shrimp allergy in a subject, the method comprising: (i) performing an IgE binding assay on a serum or plasma sample obtained from the subject using one or more shrimp allergens; and (ii) performing an IgE crosslinking assay on a blood sample obtained from the subject using shrimp TnC and/or FABP; wherein a detection in step (i) of a greater amount of IgE binding to the one or more shrimp allergens in the serum or plasma sample as compared to the amount in serum or plasma from a non-allergic control, and a detection in step (ii) of a greater amount of IgE crosslinking in immune cells in the blood sample in the presence of the shrimp TnC and/or FABP as compared to the amount in blood from a non-allergic control, indicates that that subject has a shrimp allergy.

In some embodiments of the method, the IgE-binding assay is performed using a shrimp extract. In some embodiments, the IgE binding assay is performed using shrimp TnC and/or FABP. In some embodiments, the IgE binding assay is an ELISA or ImmunoCAP assay. In some embodiments, the IgE crosslinking assay is a BAT. In some embodiments, the BAT is performed using shrimp FABP. In some embodiments, basophil activation is detected by detecting increased numbers of CD63⁺ basophils in the blood sample. In some embodiments, the one or more shrimp antigens, the shrimp TnC, and/or the shrimp FABP is from Penaeus monodon. In some embodiments, the method further comprises performing a skin-prick test on the subject using one or more shrimp allergens. In some embodiments, the skin-prick test is performed using shrimp TnC and/or shrimp FABP. In some embodiments, the TnC and/or FABP are recombinant. In some embodiments, the subject is determined to have a shrimp allergy based on steps (i) and (ii), and the method further comprises a step in which the subject is administered a treatment for shrimp allergy. In some embodiments, the treatment comprises administering an antihistamine or a corticosteroid, or immunotherapy. In some embodiments, the antihistamine or corticosteroid is given by way of oral administration.

In another aspect, the present disclosure provides an expression cassette comprising a polynucleotide encoding Penaeus monodon TnC or FABP or a fragment thereof, operably linked to a heterologous promoter. In some embodiments, the promoter is an IPTG-inducible promoter. In some embodiments, the expression cassette further comprises a polyA tail at the 3′ end of the polynucleotide encoding the TnC or FABP or fragment thereof.

In another aspect, the present disclosure provides a plasmid or vector comprising any of the herein-described expression cassettes. In another aspect, the present disclosure provides a bacterial cell comprising any of the herein-described expression cassettes, plasmids, or vectors. In some embodiments, the bacterial cell is E. coli. In another aspect, the present disclosure provides a bacterial cell comprising a polynucleotide encoding Penaeus monodon TnC or FABP. In some embodiments, the bacterial cell is E. coli.

### BRIEF DESCRIPTION OF THE DRAWINGS

FIG. 1. SDS-PAGE of purified TnC and FABP. Purity of recombinant TnC and FABP was >95% and the estimated protein sizes were 25 kDa and 20 kDa, respectively.

FIG. 2. IgE binding reactivity of TnC as shown by ELISA. Recombinant TnC was coated in 96-well ELISA plates at 5 μg/mL and incubated with sera from shrimp allergic (n=30), shrimp tolerant (n=16), and house dust mite (HDM) sensitized control (n=18) subjects at 1:10 dilution. Signal was acquired at OD 450 nm and the threshold was set at 0.2. 22/30 (73.3%) shrimp allergic subjects showed positive IgE binding to TnC, while none of the subjects from the tolerant and HDM control groups showed IgE binding. By One-way ANOVA, the TnC-specific IgE level in the allergic group was also statistically higher than that of the HDM control and tolerant groups.

FIG. 3. IgE binding reactivity of FABP as shown by ELISA. Recombinant FABP was coated in 96-well ELISA plates at 5 μg/mL and incubated with sera from shrimp allergic (n=30), shrimp tolerant (n=16), and house dust mite (HDM) sensitized control (n=18) subjects at 1:10 dilution. Signal was acquired at OD 450 nm and the threshold was set at 0.2. 20/30 (66.7%) shrimp allergic subjects showed positive IgE binding to FABP, while none of the subjects from the tolerant and HDM control groups showed IgE binding. By One-way ANOVA, the FABP-specific IgE level in the allergic group was also statistically higher than that of the HDM control and tolerant groups.

FIG. 4. ROC curves plotting the proportion of the allergic and tolerant subjects. The curve for each diagnostic marker, including shrimp extract, tropomyosin (TM), TnC, and FABP is depicted by a different symbol. AUC values (95% CI) are shown. Note that the AUC values for sIgE levels to TnC and FABP are higher than that to shrimp extract and TM, indicating the higher diagnostic values of TnC and FABP as compared to other markers.

FIG. 5. IgE crosslinking reactivity of TnC as shown by the basophil activation test. Recombinant TnC was used as a stimulant at 10,000 ng/mL to stimulate basophils from shrimp allergic (n=12) and tolerant subjects (n=14), as confirmed by double-blind placebo-controlled food challenge (DBPCFC) using the Flow CAST kit (BÜHLMANN Laboratories). The % CD63⁺ basophils was measured on the BD LSRFORTESSA flow cytometer. Basophils of 7/12 shrimp allergic subjects were activated by recombinant TnC (i.e., >9% CD63⁺ basophils), while only one in the tolerant group showed a positive response.

FIG. 6. IgE crosslinking reactivity of FABP as shown by the basophil activation test. Recombinant FABP was used as stimulant at 10,000 ng/mL to stimulate basophils from shrimp allergic (n=12) and tolerant subjects (n=14) as confirmed by DBPCFC using the Flow CAST kit (BUHLMANN Laboratories). The % CD63⁺ basophils was measured on the BD LSRFORTESSA flow cytometer. Basophils of 7/12 shrimp allergic subjects were activated by recombinant FABP (i.e., >9% CD63⁺ basophils), while none in the tolerant group showed a positive response.

FIG. 7. ROC curves plotting the proportion of the allergic and tolerant subjects. The curves for measuring IgE level and basophil activation to TnC and FABP are depicted by different symbols, as indicated. AUC values (95% CI) are shown. Note that the AUC values for measuring basophil activation against FABP and TnC are higher than those for measuring the respective IgE levels, indicating the improved diagnostic value of FABP and TnC when employed in crosslinking-based diagnostic assays.

FIGS. 8A-8B. Stepwise diagnostic approach. FIG. 8A: Conventional diagnostic steps using skin prick test (SPT) (≥3 mm wheal size cut-off) and sIgE level to shrimp (≥0.35 kUA/L). FIG. 8B: Incorporating the BAT assay with FABP (BAT-FABP) as the second diagnostic step after detecting sIgE level to shrimp ((≥0.35 kUA/L).

### DETAILED DESCRIPTION OF THE INVENTION

1. Introduction

The present invention provides improved methods and compositions for diagnosing and treating shrimp allergies. The invention involves the use of two novel shrimp allergens, troponin C (TnC) and fatty-acid binding protein (FABP), for shrimp allergy diagnosis. The two allergens can be cloned, expressed, and purified as recombinant proteins, and their IgE reactivities validated, e.g., with sera of shrimp allergic subjects and non-allergic controls. Immunological assays, including the IgE-binding test and basophil activation test (BAT), indicate the superior diagnostic accuracy of TnC and FABP for detecting shrimp allergy.

The present invention provides diagnostic methods with enhanced diagnostic accuracy of shrimp allergy over current methods. This novel approach uses newly identified markers that are particularly relevant to Asia-Pacific populations, and can reduce the frequency of false-positive results and provide improved sensitivity and specificity. The incorporation of these markers into the diagnostic workflow can also reduce unnecessary oral food challenges when discriminating between shrimp allergic and tolerant subjects. Accordingly, the present disclosure provides methods to produce shrimp TnC and FABP, e.g., by recombinant or synthetic means, and incorporate them into existing allergy assays and diagnostic workflow for shrimp allergy. The methods can be adopted as a first-line diagnostic test, or incorporated into other diagnostic workflows for shrimp allergy in clinical practice.

2. Definitions

As used herein, the following terms have the meanings ascribed to them unless specified otherwise.

The terms “a,” “an,” or “the” as used herein not only include aspects with one member, but also include aspects with more than one member. For instance, the singular forms “a,” “an,” and “the” include plural referents unless the context clearly dictates otherwise. Thus, for example, reference to “a cell” includes a plurality of such cells and reference to “the agent” includes reference to one or more agents known to those skilled in the art, and so forth.

The terms “about” and “approximately” as used herein shall generally mean an acceptable degree of error for the quantity measured given the nature or precision of the measurements. Typically, exemplary degrees of error are within 20 percent (%), preferably within 10%, and more preferably within 5% of a given value or range of values. Any reference to “about X” specifically indicates at least the values X, 0.8X, 0.81X, 0.82X, 0.83X, 0.84X, 0.85X, 0.86X, 0.87X, 0.88X, 0.89X, 0.9X, 0.91X, 0.92X, 0.93X, 0.94X, 0.95X, 0.96X, 0.97X, 0.98X, 0.99X, 1.01X, 1.02X, 1.03X, 1.04X, 1.05X, 1.06X, 1.07X, 1.08X, 1.09X, 1.1X, 1.11X, 1.12X, 1.13X, 1.14X, 1.15X, 1.16X, 1.17X, 1.18X, 1.19X, and 1.2X. Thus, “about X” is intended to teach and provide written description support for a claim limitation of, e.g., “0.98X.”

“Troponin C” (TnC) is a protein in the troponin complex that is responsible for binding calcium to activate the contraction of striated muscles. As used herein, troponin C refers in particular to troponin C from shrimp, and most specifically to troponin C from Penaeus monodon. The protein sequence of TnC can be found, e.g., on the Uniprot database (UniProt ID:E7CGC5, the entire disclosure of which is herein incorporated by reference), and Troponin C as referred to herein can refer to any polypeptide comprising the amino acid sequence of UniProt ID:E7CGC5, or to polypeptides comprising an amino acid sequence that is at least 80%, 85%, 90%, 95%, 96%, 97%, 98%, 99%, or more identical to the amino acid sequence of UniProt ID:E7CGC5, or to derivatives and/or fragments thereof. A cDNA sequence encoding Troponin C is shown, e.g., as NCBI GenBank No. HM034316.1, the entire disclosure of which is herein incorporated by reference, and Troponin C can comprise the nucleotide sequence of NCBI GenBank No. HM034316.1, or a nucleotide sequence that is at least 80%, 8%, 90%, 95%, 96%, 97%, 98%, 99%, or more identical to NCBI GenBank No. HM034316.1, as well as to fragments and derivatives, e.g., codon-optimized derivatives, thereof. In addition, Troponin C comprises nucleotide sequences that encode UniProt ID:E7CGC5, or that encode a polypeptide that is at least 80%, 85%, 90%, 95%, 96%, 97%, 98%, 99%, or more identical to UniProt ID:E7CGC5, or to derivatives and/or fragments thereof. TnC of the invention can be isolated/purified from natural sources, can be chemically synthesized, or can be recombinantly produced.

“Fatty acid binding protein” (FABP) is a protein that transports fatty acids and other lipophilic substances in cells. As used herein, FABP refers in particular to FABP from shrimp, and most specifically to FABP from Penaeus monodon. The protein sequence of FABP can be found, e.g., on the Uniprot database (UniProt ID: Q1KS35, the entire disclosure of which is herein incorporated by reference), and FABP as referred to herein can refer to any polypeptide comprising the amino acid sequence of UniProt ID: Q1KS35, or to polypeptides comprising an amino acid sequence that is at least 80%, 85%, 90%, 95%, 96%, 97%, 98%, 99%, or more identical to the amino acid sequence of UniProt ID: Q1KS35, or to derivatives and/or fragments thereof. A cDNA sequence encoding FABP is shown, e.g., as NCBI GenBank No. JN572542.1, the entire disclosure of which is herein incorporated by reference, and FABP can comprise the nucleotide sequence of NCBI GenBank No. JN572542.1, or a nucleotide sequence that is at least 80%, 8%, 90%, 95%, 96%, 97%, 98%, 99%, or more identical to NCBI GenBank No. JN572542.1, as well as to fragments and derivatives, e.g., codon-optimized derivatives, thereof. In addition, FABP comprises nucleotide sequences that encode UniProt ID: Q1KS35, or that encode a polypeptide that is at least 80%, 85%, 90%, 95%, 96%, 97%, 98%, 99%, or more identical to UniProt ID: Q1KS35, or to derivatives and/or fragments thereof. FABP of the invention can be isolated/purified from natural sources, can be chemically synthesized, or can be recombinantly produced.

The term “allergen” refers not only to naturally occurring allergen extracts and allergen molecules but also to mutants of allergens, hypoallergens or parts of allergen molecules, such as polypeptides. Allergens are able to trigger an allergy, that is, an immediate-type hypersensitivity reaction, which is induced by the synthesis of IgE antibodies. Hypoallergens are natural or recombinant derivatives of an allergen molecule which, due to slight differences compared with the amino acid sequence of the allergen, assume a conformation by which IgE-binding properties are lost.

The term “immunoassay” describes an assay that uses an antibody to specifically bind an antigen. The immunoassay is characterized by the use of specific binding properties of a particular antibody to identify, isolate, target, and/or detect the presence or quantity of the antigen. Examples of immunoassays that can be used in the present methods include, but are not limited to, ELISA and ImmunoCAP. In the present ELISA assays (enzyme-linked immunosorbent assay) an antigen, e.g., TnC or FABP, is immobilized to a solid surface and incubated in the presence of serum potentially containing IgE antibodies specific to the antigen. Following incubation, the presence of antigen-specific IgEs is detected, e.g., using labeled (e.g., biotinylated) anti-IgE antibodies, and then detecting the label using standard methods. “ImmunoCAP” (Thermo Fisher/Phadia) is another immunoassay for detecting antigen-specific IgE antibodies. Like with the ELISA assay, ImmunoCAP also uses an immobilized antigen, in this case to a cellulose sponge material, and often uses fluorescent labels.

The phrase “specifically binds,” when used to describe the binding relationship between an antibody and its target antigen, refers to a binding reaction that is determinative of the presence of the antigen (e.g., a polypeptide) in a heterogeneous population of proteins and other biologics. Thus, under designated immunoassay conditions, the specified antibodies bind to a particular polypeptide at least two times the background and do not substantially bind in a significant amount to other polypeptides or other antigens present in the sample. Specific binding to an antibody under such conditions may require an antibody that is selected for its specificity for a particular protein. For example, polyclonal antibodies raised to a particular antigen can be selected to obtain only those polyclonal antibodies that are specifically immunoreactive with that specific antigen and not with other proteins. This selection may be achieved by subtracting out antibodies that cross-react with molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988) for a description of immunoassay formats and conditions that can be used to determine specific immunoreactivity). Typically, a specific binding reaction will yield at least twice of the background signal or noise and more typically more than 10, 20, 50, or up to 100 times the background.

The term “nucleic acid sequence encoding a peptide” refers to a segment of DNA, which in some embodiments may be a gene or a portion thereof, that is involved in producing a peptide chain (e.g., an antigen). A gene will generally include regions preceding and following the coding region (leader and trailer) involved in the transcription/translation of the gene product and the regulation of the transcription/translation. A gene can also include intervening sequences (introns) between individual coding segments (exons). Leaders, trailers, and introns can include regulatory elements that are necessary during the transcription and the translation of a gene (e.g., promoters, terminators, translational regulatory sequences such as ribosome binding sites and internal ribosome entry sites, enhancers, silencers, insulators, boundary elements, replication origins, matrix attachment sites and locus control regions, etc.). A “gene product” can refer to either the mRNA or protein expressed from a particular gene.

The terms “expression” and “expressed” refer to the production of a transcriptional and/or translational product, e.g., of a nucleic acid sequence encoding a protein (e.g., an antigen). In some embodiments, the term refers to the production of a transcriptional and/or translational product encoded by a gene (e.g., a gene encoding an antigen) or a portion thereof. The level of expression of a DNA molecule in a cell may be assessed on the basis of either the amount of corresponding mRNA that is present within the cell or the amount of protein encoded by that DNA produced by the cell.

The term “promoter,” as used herein, refers to a polynucleotide sequence capable of driving transcription of a coding sequence in a cell. Thus, promoters used in the polynucleotide constructs of the invention include cis-acting transcriptional control elements and regulatory sequences that are involved in regulating or modulating the timing and/or rate of transcription of a gene. For example, a promoter can be a cis-acting transcriptional control element, including an enhancer, a promoter, a transcription terminator, an origin of replication, a chromosomal integration sequence, 5′ and 3′ untranslated regions, or an intronic sequence, which are involved in transcriptional regulation. These cis-acting sequences typically interact with proteins or other biomolecules to carry out (turn on/off, regulate, modulate, etc.) gene transcription. A “constitutive promoter” is one that is capable of initiating transcription in nearly all tissue types, whereas a “tissue-specific promoter” initiates transcription only in one or a few particular tissue types. An “inducible promoter”, such as an IPTG-inducible promoter, is one that initiates transcription only under particular environmental conditions or developmental conditions.

The term “recombinant” when used with reference, e.g., to a polynucleotide, protein, vector, or cell, indicates that the polynucleotide, protein, vector, or cell has been modified by the introduction of a heterologous nucleic acid or protein or the alteration of a native nucleic acid or protein, or that the cell is derived from a cell so modified. For example, recombinant polynucleotides contain nucleic acid sequences that are not found within the native (non-recombinant) form of the polynucleotide.

As used herein, the terms “polynucleotide,” “nucleic acid,” and “nucleotide,” refer to deoxyribonucleic acids (DNA) or ribonucleic acids (RNA) and polymers thereof. The term includes, but is not limited to, single-, double-, or multi-stranded DNA or RNA, genomic DNA, cDNA, and DNA-RNA hybrids, as well as other polymers comprising purine and/or pyrimidine bases or other natural, chemically modified, biochemically modified, non-natural, synthetic, or derivatized nucleotide bases. Unless specifically limited, the term encompasses nucleic acids containing known analogs of natural nucleotides that have similar binding properties as the reference nucleic acid. Unless otherwise indicated, a particular nucleic acid sequence also implicitly encompasses conservatively modified variants thereof (e.g., degenerate codon substitutions), homologs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

The terms “vector” and “expression vector” refer to a nucleic acid construct, generated recombinantly or synthetically, with a series of specified nucleic acid elements that permit transcription of a particular nucleic acid sequence (e.g., encoding an antigen of the invention) in a host cell or engineered cell. In some embodiments, a vector includes a polynucleotide to be transcribed, operably linked to a promoter. Other elements that may be present in a vector include those that enhance transcription (e.g., enhancers), those that terminate transcription (e.g., terminators), those that confer certain binding affinity or antigenicity to a protein (e.g., recombinant protein) produced from the vector, and those that enable replication of the vector and its packaging (e.g., into a viral particle). In some embodiments, the vector is a viral vector (i.e., a viral genome or a portion thereof). A vector may contain nucleic acid sequences or mutations, for example, that increase tropism and/or modulate immune function. An “expression cassette” refers to a coding sequence for a protein, operably linked to a promoter, and optionally including additional elements to ensure or regulate expression, e.g., a polyA tail.

The terms “polypeptide,” “peptide,” and “protein” are used interchangeably herein to refer to a polymer of amino acid residues. All three terms apply to amino acid polymers in which one or more amino acid residues are an artificial chemical mimetic of a corresponding naturally occurring amino acid, as well as to naturally occurring amino acid polymers and non-naturally occurring amino acid polymers. As used herein, the terms encompass amino acid chains of any length, including full-length proteins, wherein the amino acid residues are linked by covalent peptide bonds.

The term “amino acid” includes naturally-occurring a-amino acids and their stereoisomers, as well as unnatural (non-naturally occurring) amino acids and their stereoisomers. “Stereoisomers” of amino acids refers to mirror image isomers of the amino acids, such as L-amino acids or D-amino acids. For example, a stereoisomer of a naturally-occurring amino acid refers to the mirror image isomer of the naturally-occurring amino acid, i.e., the D-amino acid.

Naturally-occurring amino acids are those encoded by the genetic code, as well as those amino acids that are later modified, e.g., hydroxyproline, γ-carboxyglutamate and O-phosphoserine. Naturally-occurring α-amino acids include, without limitation, alanine (Ala), cysteine (Cys), aspartic acid (Asp), glutamic acid (Glu), phenylalanine (Phe), glycine (Gly), histidine (His), isoleucine (Ile), arginine (Arg), lysine (Lys), leucine (Leu), methionine (Met), asparagine (Asn), proline (Pro), glutamine (Gln), serine (Ser), threonine (Thr), valine (Val), tryptophan (Trp), tyrosine (Tyr), and combinations thereof. Stereoisomers of a naturally-occurring α-amino acids include, without limitation, D-alanine (D-Ala), D-cysteine (D-Cys), D-aspartic acid (D-Asp), D-glutamic acid (D-Glu), D-phenylalanine (D-Phe), D-histidine (D-His), D-isoleucine (D-Ile), D-arginine (D-Arg), D-lysine (D-Lys), D-leucine (D-Leu), D-methionine (D-Met), D-asparagine (D-Asn), D-proline (D-Pro), D-glutamine (D-Gln), D-serine (D-Ser), D-threonine (D-Thr), D-valine (D-Val), D-tryptophan (D-Trp), D-tyrosine (D-Tyr), and combinations thereof.

Unnatural (non-naturally occurring) amino acids include, without limitation, amino acid analogs, amino acid mimetics, synthetic amino acids, N-substituted glycines, and N-methyl amino acids in either the L- or D-configuration that function in a manner similar to the naturally-occurring amino acids. For example, “amino acid analogs” are unnatural amino acids that have the same basic chemical structure as naturally-occurring amino acids, i.e., an a carbon that is bound to a hydrogen, a carboxyl group, an amino group, but have modified R (i.e., side-chain) groups or modified peptide backbones, e.g., homoserine, norleucine, methionine sulfoxide, methionine methyl sulfonium. “Amino acid mimetics” refer to chemical compounds that have a structure that is different from the general chemical structure of an amino acid, but that functions in a manner similar to a naturally-occurring amino acid.

Amino acids may be referred to herein by either the commonly known three letter symbols or by the one-letter symbols recommended by the IUPAC-IUB Biochemical Nomenclature Commission. Nucleotides, likewise, may be referred to by their commonly accepted single-letter codes.

With respect to amino acid sequences, one of skill in the art will recognize that individual substitutions, additions, or deletions to a peptide, polypeptide, or protein sequence which alters, adds, or deletes a single amino acid or a small percentage of amino acids in the encoded sequence is a “conservatively modified variant” where the alteration results in the substitution of an amino acid with a chemically similar amino acid. The chemically similar amino acid includes, without limitation, a naturally-occurring amino acid such as an L-amino acid, a stereoisomer of a naturally occurring amino acid such as a D-amino acid, and an unnatural amino acid such as an amino acid analog, amino acid mimetic, synthetic amino acid, N-substituted glycine, and N-methyl amino acid.

Conservative substitution tables providing functionally similar amino acids are well known in the art. For example, substitutions may be made wherein an aliphatic amino acid (e.g., G, A, I, L, or V) is substituted with another member of the group. Similarly, an aliphatic polar-uncharged group such as C, S, T, M, N, or Q, may be substituted with another member of the group; and basic residues, e.g., K, R, or H, may be substituted for one another. In some embodiments, an amino acid with an acidic side chain, e.g., E or D, may be substituted with its uncharged counterpart, e.g., Q or N, respectively; or vice versa. Each of the following eight groups contains other exemplary amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

The term “amino acid modification” or “amino acid alteration” refers to a substitution, a deletion, or an insertion of one or more amino acids.

The terms “subject,” “individual,” and “patient” are used interchangeably herein to refer to a vertebrate, preferably a mammal, more preferably a human. Mammals include, but are not limited to, murines, mice, rats, simians, humans, farm animals, sport animals, and pets. Tissues, cells and their progeny of a biological entity obtained in vivo or cultured in vitro are also encompassed.

As used herein, the term “administering” includes oral administration, topical contact, administration as a suppository, intravenous, intraperitoneal, intramuscular, intralesional, intratumoral, intrathecal, intranasal, intraosseous, or subcutaneous administration to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arterial, intradermal, subcutaneous, intraperitoneal, intraventricular, intraosseous, and intracranial. Other modes of delivery include, but are not limited to, the use of liposomal formulations, intravenous infusion, transdermal patches, etc.

The term “treating” refers to an approach for obtaining beneficial or desired results including, but not limited to, a therapeutic benefit and/or a prophylactic benefit. “Therapeutic benefit” means any therapeutically relevant improvement in or effect on one or more diseases, conditions, or symptoms under treatment. Therapeutic benefit can also mean to effect a cure of one or more diseases, conditions, or symptoms under treatment. Furthermore, therapeutic benefit can also mean to increase survival. For prophylactic benefit, the compositions may be administered to a subject at risk of developing a particular disease, condition, or symptom, or to a subject reporting one or more of the physiological symptoms of a disease, even though the disease, condition, or symptom may not yet be present.

The term “therapeutically effective amount” or “sufficient amount” refers to the amount of a system, recombinant polynucleotide, or composition described herein that is sufficient to effect beneficial or desired results. The therapeutically effective amount may vary depending upon one or more of: the subject and disease condition being treated, the weight and age of the subject, the severity of the disease condition, the immune status of the subject, the manner of administration and the like, which can readily be determined by one of ordinary skill in the art. The specific amount may vary depending on one or more of: the particular agent chosen, the target cell type, the location of the target cell in the subject, the dosing regimen to be followed, whether it is administered in combination with other compounds, timing of administration, and the physical delivery system in which it is carried.

For the purposes herein an effective amount is determined by such considerations as may be known in the art. The amount must be effective to achieve the desired therapeutic effect in a subject suffering from a condition such as an allergy. The desired therapeutic effect may include, for example, amelioration of undesired symptoms associated with the condition, prevention of the manifestation of such symptoms before they occur, slowing down the progression of symptoms associated with the condition, slowing down or limiting any irreversible damage caused by the condition, lessening the severity of or curing the condition, or improving the survival rate or providing more rapid recovery from the condition. Further, in the context of prophylactic treatment the amount may also be effective to prevent the development of the condition.

The term “pharmaceutically acceptable carrier” refers to a substance that aids the administration of an active agent to a cell, an organism, or a subject. “Pharmaceutically acceptable carrier” also refers to a carrier or excipient that can be included in the compositions of the invention and that causes no significant adverse toxicological effect on the patient. Non-limiting examples of pharmaceutically acceptable carriers include water, sodium chloride (NaCl), normal saline solutions, lactated Ringer's, normal sucrose, normal glucose, binders, fillers, disintegrants, lubricants, coatings, sweeteners, flavors and colors, liposomes, dispersion media, microcapsules, cationic lipid carriers, isotonic and absorption delaying agents, and the like. The carrier may also comprise or consist of substances for providing the formulation with stability, sterility and isotonicity (e.g. antimicrobial preservatives, antioxidants, chelating agents and buffers), for preventing the action of microorganisms (e.g. antimicrobial and antifungal agents, such as parabens, chlorobutanol, phenol, sorbic acid and the like) or for providing the formulation with an edible flavor, etc. In some instances, the carrier is an agent that facilitates the delivery of a polypeptide or polynucleotide to a target cell or tissue. One of skill in the art will recognize that other pharmaceutical carriers are useful in the present invention.

As used in this application, an “increase” or a “decrease” refers to a detectable positive or negative change in quantity from a comparison control, e.g., an established standard control (such as an average level of IgE binding or crosslinking found in a pre-determined sample type, e.g., serum, plasma, or whole blood, from healthy subjects who do not have shrimp allergy). An increase is a positive change that is typically at least 10%, or at least 20%, or 50%, or 100%, and can be as high as at least 2-fold or at least 5-fold or even 10-fold of the control value. Similarly, a decrease is a negative change that is typically at least 10%, or at least 20%, 30%, or 50%, or even as high as at least 80% or 90% of the control value. Other terms indicating quantitative changes or differences from a comparative basis, such as “greater,” “more,” “less,” “higher,” and “lower,” are used in this application in the same fashion as described above. In contrast, the term “substantially the same” or “substantially lack of change” indicates little to no change in quantity from the standard control value, typically within ±10% of the standard control, or within ±5%, 2%, or even less variation from the standard control.

In this disclosure the term “blood sample” includes blood and blood fractions or products, e.g., whole blood, acellular fraction of blood—serum or plasma, and cellular fraction of blood—blood cells.

3. Allergens

The present methods and compositions provide improvements over existing methods of diagnosis of shrimp allergies, particularly for individuals from Asia-Pacific populations. In particular, the methods and composition involve shrimp TnC and shrimp FABP. It has been discovered that these proteins can be detected using, e.g., IgE-binding assays, IgE crosslinking assays, or skin prick tests, and thereby allow accurate diagnostic methods with reduced frequencies of false-positive results, and improved sensitivity and specificity. For example, these proteins can be marketed globally for the clinical diagnosis of shrimp allergy on platforms such as ImmunoCAP, ISAC and ALEX, as well as for SPT, BAT and other IgE-crosslinking assays reagents.

TnC and FABP from any shrimp species can be used in the methods. They may be obtained by isolation/purification of naturally occurring proteins, by chemical synthesis, or by recombinant production. In particular embodiments, the TnC and/or FABP is from Penaeus monodon. The protein sequences of TnC and FABP from Penaeus monodon can be found, e.g., on the Uniprot database (UniProt ID:E7CGC5 and Q1KS35, respectively), and the nucleotide sequences can be obtained by reverse translation (e.g., using MEGA 7.0) or through sequence databases (e.g., NCBI GenBank Nos. HM034316.1 or JN572542.1, respectively), or through aligning protein sequences to a nucleotide database (e.g., TBASTN). Immunogenic fragments, variants, and derivatives of shrimp TnC and/or FABP can be used as well.

4. Recombinant production of the allergens

The synthesis of shrimp TnC and/or FABP for use in the present methods can be accomplished using standard molecular biology methods. For example, the nucleotide sequences coding full-length TnC and FABP can be synthesized using standard methods and cloned into a suitable expression vector, e.g., the His-tag expression vector pET30(a)+. Recombinant TnC and FABP can then be expressed in suitable cells, e.g., E. coli, and purified, and the protein concentrations and purities determined by, e.g., BCA assay and SDS-PAGE, respectively.

Basic texts disclosing general methods and techniques in the field of recombinant genetics include Sambrook and Russell, Molecular Cloning, A Laboratory Manual (3rd ed. 2001); Kriegler, Gene Transfer and Expression: A Laboratory Manual (1990); and Ausubel et al., eds., Current Protocols in Molecular Biology (1994).

For nucleic acids, sizes are given in either kilobases (kb) or base pairs (bp). These are estimates derived from agarose or acrylamide gel electrophoresis, from sequenced nucleic acids, or from published DNA sequences. For proteins, sizes are given in kilodaltons (kDa) or amino acid residue numbers. Proteins sizes are estimated from gel electrophoresis, from sequenced proteins, from derived amino acid sequences, or from published protein sequences.

Oligonucleotides that are not commercially available can be chemically synthesized, e.g., according to the solid phase phosphoramidite triester method first described by Beaucage & Caruthers, Tetrahedron Lett. 22: 1859-1862 (1981), using an automated synthesizer, as described in Van Devanter et. al., Nucleic Acids Res. 12: 6159-6168 (1984). Purification of oligonucleotides is performed using any art-recognized strategy, e.g., native acrylamide gel electrophoresis or anion-exchange HPLC as described in Pearson & Reanier, J. Chrom. 255: 137-149 (1983).

The sequence of a polynucleotide encoding an allergen of this invention (e.g., Penaeus monodon TnC and FABP) can be verified after cloning or subcloning using, e.g., the chain termination method for sequencing double-stranded templates of Wallace et al., Gene 16: 21-26 (1981).

Polynucleotide sequences encoding the allergens of this invention can be determined based on their amino acid sequences (e.g., as shown in UniProt ID:E7CGC5 and Q1KS35) and available information of the Penaeus monodon genome. They can be isolated from a Penaeus monodon cDNA or genomic library or can be synthesized by a commercial supplier. Nucleic acid sequences encoding the allergens of this invention can be isolated using standard cloning techniques such as polymerase chain reaction (PCR). Most commonly used techniques for this purpose are described in standard texts, e.g., Sambrook and Russell, supra.

cDNA libraries suitable for obtaining a coding sequence for an allergen of this invention may be commercially available or can be constructed. The general methods of isolating mRNA, making cDNA by reverse transcription, ligating cDNA into a recombinant vector, transfecting into a recombinant host for propagation, screening, and cloning are well known (see, e.g., Gubler and Hoffman, Gene, 25: 263-269 (1983); Ausubel et al., supra). Upon obtaining an amplified segment of nucleotide sequence by PCR, the segment can be further used as a probe to isolate a longer length polynucleotide sequence encoding the allergen from the cDNA library. A general description of appropriate procedures can be found in Sambrook and Russell, supra.

Based on sequence homology, degenerate oligonucleotides can be designed as primer sets and PCR can be performed under suitable conditions (see, e.g., White et al., PCR Protocols: Current Methods and Applications, 1993; Griffin and Griffin, PCR Technology, CRC Press Inc. 1994) to amplify a segment of nucleotide sequence from a cDNA or genomic library. Using the amplified segment as a probe, a longer length nucleic acid encoding an allergen of this invention is obtained.

Upon acquiring a nucleic acid sequence encoding an allergen of this invention, the coding sequence can be modified as appropriate (e.g., adding a coding sequence for a heterologous tag, such as an affinity tag, for example, 6×His tag or GST tag) and then be subcloned into a vector, for instance, an expression vector, so that a recombinant allergen can be produced from the resulting construct, for example, after transfection and culturing host cells under conditions permitting recombinant protein expression directed by a promoter operably linked to the coding sequence.

In some embodiments, the polynucleotide sequence encoding an allergen of the invention can be further altered to coincide with the preferred codon usage of a particular host. For example, the preferred codon usage of one strain of bacterial cells can be used to derive a polynucleotide that encodes an allergen of this invention and includes the codons favored by this strain. The frequency of preferred codon usage exhibited by a host cell can be calculated by averaging frequency of preferred codon usage in a large number of genes expressed by the host cell (e.g., calculation service is available from web site of the Kazusa DNA Research Institute, Japan). This analysis is preferably limited to genes that are highly expressed by the host cell.

Chemical synthesis

The polypeptide allergens of the invention, e.g., Penaeus monodon TnC and FABP and immunogenic fragments thereof, can also be synthesized chemically using peptide synthesis or other protocols well known in the art.

Polypeptides may also be synthesized by solid-phase peptide synthesis methods using procedures similar to those described by Merrifield et al., J. Am. Chem. Soc., 85:2149-2156 (1963); Barany and Merrifield, Solid-Phase Peptide Synthesis, in The Peptides: Analysis, Synthesis, Biology Gross and Meienhofer (eds.), Academic Press, N.Y., vol. 2, pp. 3-284 (1980); and Stewart et al., Solid Phase Peptide Synthesis 2nd ed., Pierce Chem. Co., Rockford, Ill. (1984). During synthesis, N-α-protected amino acids having protected side chains are added stepwise to a growing polypeptide chain linked by its C-terminal and to a solid support, i.e., polystyrene beads. The peptides are synthesized by linking an amino group of an N-α-deprotected amino acid to an α-carboxy group of an N-α-protected amino acid that has been activated by reacting it with a reagent such as dicyclohexylcarbodiimide. The attachment of a free amino group to the activated carboxyl leads to peptide bond formation. The most commonly used N-α-protecting groups include Boc, which is acid labile, and Fmoc, which is base labile.

Expression of recombinant allergens

To obtain high level expression of a nucleic acid encoding an allergen of the present invention, a polynucleotide encoding the polypeptide can be subcloned into an expression vector that contains a strong promoter (typically heterologous, i.e., of non-Penaeus monodon origin, and/or not naturally linked to the coding sequence for the allergen) to direct transcription, a transcription/translation terminator and a ribosome binding site for translational initiation. Suitable bacterial promoters are well known in the art and described, e.g., in Sambrook and Russell, supra, and Ausubel et al., supra. Bacterial expression systems for expressing a recombinant polypeptide are available in, e.g., E. coli, Bacillus sp., Salmonella, and Caulobacter. Kits for such expression systems are commercially available. Eukaryotic expression systems for mammalian cells, yeast, and insect cells are well known in the art and are also commercially available. In one embodiment, the eukaryotic expression vector is an adenoviral vector, an adeno-associated vector, or a retroviral vector.

The promoter used to direct expression of a heterologous nucleic acid depends on the particular application. The promoter is optionally positioned about the same distance from the heterologous transcription start site as it is from the transcription start site in its natural setting. As is known in the art, however, some variation in this distance can be accommodated without loss of promoter function. In one embodiment, the promoter is an IPTG-inducible promoter.

In addition to the promoter, the expression vector typically includes a transcription unit or expression cassette that contains all the additional elements required for the expression of the allergen in host cells. A typical expression cassette thus contains a promoter operably linked to the coding sequence and signals required for efficient polyadenylation of the transcript, ribosome binding sites, and translation termination. The nucleic acid sequence encoding the allergen is typically linked to a cleavable signal peptide sequence to promote secretion of the recombinant polypeptide by the transformed cell. Such signal peptides include, among others, the signal peptides from tissue plasminogen activator, insulin, and neuron growth factor, and juvenile hormone esterase of Heliothis virescens. Additional elements of the cassette may include enhancers and, if genomic DNA is used as the structural gene, introns with functional splice donor and acceptor sites.

In addition to a promoter sequence, the expression cassette should also contain a transcription termination region downstream of the structural gene to provide for efficient termination. The termination region may be obtained from the same gene as the promoter sequence or may be obtained from a different genes.

The particular expression vector used to transport the genetic information into the cell is not particularly critical. Any of the conventional vectors used for expression in eukaryotic or prokaryotic cells may be used. Standard bacterial expression vectors include plasmids such as pBR322 based plasmids, pSKF, pET23D, pET30(a)+, and fusion expression systems such as GST and LacZ. Epitope tags can also be added to recombinant proteins to provide convenient methods of isolation, e.g., c-myc.

Expression vectors containing regulatory elements from eukaryotic viruses are typically used in eukaryotic expression vectors, e.g., SV40 vectors, papilloma virus vectors, and vectors derived from Epstein-Barr virus. Other exemplary eukaryotic vectors include pMSG, pAV009/A⁺, pMTO10/A⁺, pMAMneo-5, baculovirus pDSVE, and any other vector allowing expression of proteins under the direction of the SV40 early promoter, SV40 later promoter, metallothionein promoter, murine mammary tumor virus promoter, Rous sarcoma virus promoter, polyhedrin promoter, or other promoters shown effective for expression in eukaryotic cells.

Some expression systems have markers that provide gene amplification such as thymidine kinase, hygromycin B phosphotransferase, and dihydrofolate reductase. Alternatively, high yield expression systems not involving gene amplification are also suitable, such as a baculovirus vector in insect cells, with a polynucleotide sequence encoding the allergen under the direction of the polyhedrin promoter or other strong baculovirus promoters.

The elements that are typically included in expression vectors also include a replicon that functions in E. coli, a gene encoding a protein that provides antibiotic resistance to permit selection of bacteria that harbor recombinant plasmids, and unique restriction sites in nonessential regions of the plasmid to allow insertion of eukaryotic sequences. The particular antibiotic resistance gene chosen is not critical, any of the many resistance genes known in the art are suitable. The prokaryotic sequences are optionally chosen such that they do not interfere with the replication of the DNA in eukaryotic cells, if necessary. Similar to antibiotic resistance selection markers, metabolic selection markers based on known metabolic pathways may also be used as a means for selecting transformed host cells.

When periplasmic expression of a recombinant protein (e.g., a TnC or FABP polypeptide of the present invention) is desired, the expression vector further comprises a sequence encoding a secretion signal, such as the E. coli OppA (Periplasmic Oligopeptide Binding Protein) secretion signal or a modified version thereof, which is directly connected to 5′ of the coding sequence of the protein to be expressed. This signal sequence directs the recombinant protein produced in cytoplasm through the cell membrane into the periplasmic space. The expression vector may further comprise a coding sequence for signal peptidase 1, which is capable of enzymatically cleaving the signal sequence when the recombinant protein is entering the periplasmic space. More detailed description for periplasmic production of a recombinant protein can be found in, e.g., Gray et al., Gene 39: 247-254 (1985), U.S. Pat. Nos. 6,160,089 and 6,436,674.

Transfection

Standard transfection methods are used to produce bacterial, mammalian, yeast, insect, or plant cell lines that express large quantities of a recombinant polypeptide, which are then purified using standard techniques (see, e.g., Colley et al., J. Biol. Chem. 264: 17619-17622 (1989); Guide to Protein Purification, in Methods in Enzymology, vol. 182 (Deutscher, ed., 1990)). Transformation of eukaryotic and prokaryotic cells are performed according to standard techniques (see, e.g., Morrison, J. Bact. 132: 349-351 (1977); Clark-Curtiss & Curtiss, Methods in Enzymology 101: 347-362 (Wu et al., eds, 1983).

Any of the well-known procedures for introducing foreign nucleotide sequences into host cells may be used. These include the use of calcium phosphate transfection, polybrene, protoplast fusion, electroporation, liposomes, microinjection, plasma vectors, viral vectors and any of the other well-known methods for introducing cloned genomic DNA, cDNA, synthetic DNA, or other foreign genetic material into a host cell (see, e.g., Sambrook and Russell, supra). It is only necessary that the particular genetic engineering procedure used be capable of successfully introducing at least one gene into the host cell capable of expressing the recombinant polypeptide.

Detection of expression in host cells

After the expression vector is introduced into appropriate host cells, the transfected cells are cultured under conditions favoring expression of the allergen. The cells are then screened for the expression of the recombinant polypeptide, which is subsequently recovered from the culture using standard techniques (see, e.g., Scopes, Protein Purification: Principles and Practice (1982); U.S. Pat. No. 4,673,641; Ausubel et al., supra; and Sambrook and Russell, supra).

Several general methods for screening gene expression are well known among those skilled in the art. First, gene expression can be detected at the nucleic acid level. A variety of methods of specific DNA and RNA measurement using nucleic acid hybridization techniques are commonly used (e.g., Sambrook and Russell, supra). Some methods involve an electrophoretic separation (e.g., Southern blot for detecting DNA and Northern blot for detecting RNA), but detection of DNA or RNA can be carried out without electrophoresis as well (such as by dot blot). The presence of nucleic acid encoding an allergen in transfected cells can also be detected by PCR or RT-PCR using sequence-specific primers.

Second, gene expression can be detected at the polypeptide level. Various immunological assays are routinely used by those skilled in the art to measure the level of a gene product, particularly using polyclonal or monoclonal antibodies that react specifically with an allergen of this invention (e.g., Harlow and Lane, Antibodies, A Laboratory Manual, Chapter 14, Cold Spring Harbor, 1988; Kohler and Milstein, Nature, 256: 495-497 (1975)). Such techniques require antibody preparation by selecting antibodies with high specificity against the allergen. The methods of raising polyclonal and monoclonal antibodies are well established and their descriptions can be found in the literature, see, e.g., Harlow and Lane, supra; Kohler and Milstein, Eur. J. Immunol., 6: 511-519 (1976).

Purification of Recombinantly Produced Allergens

Once the expression of a recombinant allergen of this invention in transfected host cells is confirmed, the host cells are then cultured in an appropriate scale for the purpose of purifying the recombinant polypeptide.

When the allergens of the present invention are produced recombinantly by transformed bacteria in large amounts, typically after promoter induction, although expression can be constitutive, the polypeptides may form insoluble aggregates. There are several protocols that are suitable for purification of protein inclusion bodies. For example, purification of aggregate proteins (hereinafter referred to as inclusion bodies) typically involves the extraction, separation and/or purification of inclusion bodies by disruption of bacterial cells, e.g., by incubation in a buffer of about 100-150 μg/ml lysozyme and 0.1% Nonidet P40, a non-ionic detergent. The cell suspension can be ground using a Polytron grinder (Brinkman Instruments, Westbury, N.Y.). Alternatively, the cells can be sonicated on ice. Alternate methods of lysing bacteria are described in Ausubel et al. and Sambrook and Russell, both supra, and will be apparent to those of skill in the art.

The cell suspension is generally centrifuged and the pellet containing the inclusion bodies resuspended in buffer which does not dissolve but washes the inclusion bodies, e.g., 20 mM Tris-HCl (pH 7.2), 1 mM EDTA, 150 mM NaCl and 2% Triton-X 100, a non-ionic detergent. It may be necessary to repeat the wash step to remove as much cellular debris as possible. The remaining pellet of inclusion bodies may be resuspended in an appropriate buffer (e.g., 20 mM sodium phosphate, pH 6.8, 150 mM NaCl). Other appropriate buffers will be apparent to those of skill in the art.

Following the washing step, the inclusion bodies are solubilized by the addition of a solvent that is both a strong hydrogen acceptor and a strong hydrogen donor (or a combination of solvents each having one of these properties). The proteins that formed the inclusion bodies may then be renatured by dilution or dialysis with a compatible buffer. Suitable solvents include, but are not limited to, urea (from about 4 M to about 8 M), formamide (at least about 80%, volume/volume basis), and guanidine hydrochloride (from about 4 M to about 8 M). Some solvents that are capable of solubilizing aggregate-forming proteins, such as SDS (sodium dodecyl sulfate) and 70% formic acid, may be inappropriate for use in this procedure due to the possibility of irreversible denaturation of the proteins, accompanied by a lack of immunogenicity and/or activity. Although guanidine hydrochloride and similar agents are denaturants, this denaturation is not irreversible and renaturation may occur upon removal (by dialysis, for example) or dilution of the denaturant, allowing re-formation of the immunologically and/or biologically active protein of interest. After solubilization, the protein can be separated from other bacterial proteins by standard separation techniques. For further description of purifying recombinant polypeptides from bacterial inclusion body, see, e.g., Patra et al., Protein Expression and Purification 18: 182-190 (2000).

Alternatively, it is possible to purify recombinant polypeptides, e.g., recombinant TnC or FABP, from bacterial periplasm. Where the recombinant protein is exported into the periplasm of the bacteria, the periplasmic fraction of the bacteria can be isolated by cold osmotic shock in addition to other methods known to those of skill in the art (see e.g., Ausubel et al., supra). To isolate recombinant proteins from the periplasm, the bacterial cells are centrifuged to form a pellet. The pellet is resuspended in a buffer containing 20% sucrose. To lyse the cells, the bacteria are centrifuged and the pellet is resuspended in ice-cold 5 mM MgSO₄ and kept in an ice bath for approximately 10 minutes. The cell suspension is centrifuged and the supernatant decanted and saved. The recombinant proteins present in the supernatant can be separated from the host proteins by standard separation techniques well known to those of skill in the art.

Protein Separation Techniques for Purification

When a recombinant polypeptide is expressed in host cells in a soluble form, its purification can follow a standard protein purification procedure as described herein. Such standard purification procedures are also suitable for purifying a polypeptide obtained from chemical synthesis.

Solubility Fractionation

Often as an initial step, and if the protein mixture is complex, an initial salt fractionation can separate many of the unwanted host cell proteins (or proteins derived from the cell culture media) from the recombinant protein of interest. The preferred salt is ammonium sulfate. Ammonium sulfate precipitates proteins by effectively reducing the amount of water in the protein mixture. Proteins then precipitate on the basis of their solubility. The more hydrophobic a protein is, the more likely it is to precipitate at lower ammonium sulfate concentrations. A typical protocol is to add saturated ammonium sulfate to a protein solution so that the resultant ammonium sulfate concentration is between 20-30%. This will precipitate the most hydrophobic proteins. The precipitate is discarded (unless the protein of interest is hydrophobic) and ammonium sulfate is added to the supernatant to a concentration known to precipitate the protein of interest. The precipitate is then solubilized in buffer and the excess salt removed if necessary, through either dialysis or diafiltration. Other methods that rely on solubility of proteins, such as cold ethanol precipitation, are well known to those of skill in the art and can be used to fractionate complex protein mixtures.

Size Differential Filtration

Based on a calculated molecular weight, a protein of greater and lesser size can be isolated using ultrafiltration through membranes of different pore sizes (for example, Amicon or Millipore membranes). As a first step, the protein mixture is ultrafiltered through a membrane with a pore size that has a lower molecular weight cut-off than the molecular weight of a protein of interest, e.g., shrimp TnC or FABP. The retentate of the ultrafiltration is then ultrafiltered against a membrane with a molecular cut off greater than the molecular weight of the protein of interest. The recombinant protein will pass through the membrane into the filtrate. The filtrate can then be chromatographed as described below.

Column Chromatography

The proteins of interest (such as a shrimp TnC or FABP protein of the present invention) can also be separated from other proteins on the basis of their size, net surface charge, hydrophobicity, or affinity for ligands. In addition, antibodies raised against shrimp TnC or FABP can be conjugated to column matrices and the corresponding allergen immunopurified. All of these methods are well known in the art. It will be apparent to one of skill that chromatographic techniques can be performed at any scale and using equipment from many different manufacturers (e.g., Pharmacia Biotech).

5. Assays for allergy detection

One aspect of this invention provides immunoassays used in the detection of antibodies, especially IgE and IgG antibodies, that are specifically reactive with shrimp TnC and/or FABP, e.g., for the purpose of detecting a possible case of shrimp allergy in a subject or patient who may have been exposed to shrimp and may be actively suffering from a shrimp allergy. The shrimp TnC and FABP allergens described herein are useful for carrying out these immunological assays.

Subjects

Subjects to be tested by the shrimp allergy diagnostic methods of this invention include, but are not limited to, those who have recently consumed shrimp or shrimp-containing food items or may have come into contact with shrimp, a shrimp-containing substance, or a shrimp by-product, and are exhibiting possible signs of an allergy, including but not limited to hives or a skin rash, nausea, stomach cramps, indigestion, vomiting and/or diarrhea; stuffy or runny nose and/or sneezing; headaches; asthma; and anaphylaxis. In general, however, the methods can be performed on any individual at risk of having a shrimp allergy or who wishes for any reason to determine whether they have a shrimp allergy.

In particular embodiments, to perform the herein-described immunoassays a sample is taken from a subject, e.g., a subject being tested for likely shrimp allergy. For example, a blood sample may be collected and processed (e.g., to yield a plasma or serum sample) in preparation for the specific IgE and/or IgG assay to be performed.

Immunoassays for Detecting IgE/IgG Antibodies

The amount of IgE/IgG specific for TnC or FABP in a sample, e.g., a blood/serum/plasma sample, or a skin sample or mouth swab, can be measured by a variety of immunoassay methods providing qualitative and quantitative results to a skilled artisan. For a review of immunological and immunoassay procedures in general, see, e.g., Stites, supra; U.S. Pat. Nos. 4,366,241; 4,376,110; 4,517,288; and 4,837,168. In particular embodiments, the immunoassays used to detect IgE antibodies specific to shrimp TnC and/or FABP include ELISA and ImmunoCAP.

Immunoassays often utilize a labeling agent to specifically bind to and label the binding complex formed by the antibody and the target protein (antigen). The labeling agent may itself be one of the moieties comprising the antibody/target protein complex, or may be a third moiety, such as another antibody, that specifically binds to the antibody/target protein complex. A label may be detectable by spectroscopic, photochemical, biochemical, immunochemical, electrical, optical or chemical means. Examples include, but are not limited to, magnetic beads (e.g., Dynabeads™), fluorescent dyes (e.g., fluorescein isothiocyanate, Texas red, rhodamine, and the like), radiolabels (e.g., ³H, ¹²⁵I, ³⁵S, ¹⁴C, or ³²P), enzymes (e.g., horse radish peroxidase, alkaline phosphatase, and others commonly used in an ELISA), and colorimetric labels such as colloidal gold or colored glass or plastic (e.g., polystyrene, polypropylene, latex, etc.) beads.

In some cases, the labeling agent is a second antibody (e.g., an anti-IgE or IgG antibody) bearing a detectable label. Alternatively, the second antibody may lack a label, but it may, in turn, be bound by a labeled third antibody specific to antibodies of the species from which the second antibody is derived. The second antibody can be modified with a detectable moiety, such as biotin, to which a third labeled molecule can specifically bind, such as enzyme-labeled streptavidin.

Other proteins capable of specifically binding immunoglobulin constant regions, such as protein A or protein G, can also be used as the label agents. These proteins are normal constituents of the cell walls of streptococcal bacteria. They exhibit a strong non-immunogenic reactivity with immunoglobulin constant regions from a variety of species (see, generally, Kronval, et al. J. Immunol., 111: 1401-1406 (1973); and Akerstrom, et al., J. Immunol., 135: 2589-2542 (1985)).

Immunoassays for detecting a specific IgE or IgG of interest (e.g., an IgE or IgG specifically immune-reactive against shrimp TnC or FABP) from samples may be either competitive or noncompetitive. A typical specific IgE or IgG immunoassay is a noncompetitive immunoassay in which the amount of captured target IgE or IgG is directly measured. In one preferred “sandwich” assay, for example, one or more of the allergens of the present invention can be bound or immobilized directly to a solid substrate (such as the surface of a plate). The immobilized allergen(s) can then capture the specific IgE or IgG in test samples. The antibody/target protein complex thus immobilized is then bound by a labeling agent, such as a second or third antibody bearing a label, as described above.

Other assay formats include liposome immunoassays (LIA), which use liposomes designed to bind specific molecules (e.g., antibodies) and release encapsulated reagents or markers. The released chemicals are then detected according to standard techniques (see, Monroe et al., Amer. Clin. Prod. Rev., 5: 34-41 (1986)).

For these immunoassays, the subject being tested may be one who may have been exposed to shrimp or shrimp-contaminated substance and may have begun to demonstrate symptoms of an active shrimp allergy. A positive result, indicating that the subject has or is likely to have a shrimp allergy, is obtained when more IgE binding is detected in the sample from the subject than from a control that is known to not have a shrimp allergy. The control can be an actual sample that is tested along with the sample from the subject, or a reference value that has been previously established against which the value from the subject is compared. Once a determination of likely shrimp allergy is made, the subject may be given treatment for the allergy in accordance with a physician's direction, such as administration of antihistamines or corticosteroids (e.g., administered orally) or immunotherapy (e.g., sublingual immunotherapy).

IgE crosslinking assays

In addition to “IgE binding” assays, which detect IgE antibodies in, e.g., serum, in some embodiments an “IgE crosslinking” assay is used. Such assays detect the activation or granulation of immune cells such as basophils that have IgE bound to the surface of the cells via interactions between the antibody Fc domain and Fc receptors on the cell surface. Cross-linking takes place when multiple IgE antibodies on the cell surface bind to an antigen, leading to the activation of the cell. In a particular embodiment, a basophil activation test (BAT) is used. In this assay, a blood sample is taken from a subject and incubated with an allergen, e.g., shrimp TnC and/or FABP, and the activation of basophils in the blood sample is detected by quantifying the percentage of CD63⁺ cells by FACS sorting. See, e.g., Example 3, and Sanz et al. (2002) Investig. Allergol. Clin. Immunol. 12(3):143-54; McGowan & Saini (2013) Curr Allergy Asthma Rep. 13(1): 101-109.

For IgE crosslinking assays, the subject being tested may be one who may have been exposed to shrimp or shrimp-contaminated substance and may have begun to demonstrate symptoms of an active shrimp allergy. A positive result, indicating that the subject has or is likely to have a shrimp allergy, is obtained when more IgE crosslinking, e.g., more CD63⁺ cells in a BAT test, is detected in the sample from the subject than from a control that is known to not have a shrimp allergy. The control can be an actual sample that is tested along with the sample from the subject, or a reference value that has been previously established against which the value from the subject is compared. Once a determination of likely shrimp allergy is made, the subject may be given treatment for the allergy in accordance with a physician's direction, such as administration of antihistamines or corticosteroids (e.g., administered orally) or immunotherapy (e.g., sublingual immunotherapy).

Skin prick test

In some embodiments, shrimp allergy is detected by a skin prick test (SPT) using TnC and/or FABP. For example, the protein(s) are dissolved in, e.g., glycerol/PBS, at full-strength and applied to the skin. The application step is performed immediately before or after the skin prick step. Typically, histamine and normal saline are used as positive and negative controls, respectively. The skin prick test (SPT) is performed, e.g., on the volar surface of the forearm of individual patients with single peak lancets, and the results are interpreted after a suitable amount of time, e.g., 15 min. Reactions with a wheal size of at least 3 mm are considered positive.

It will be appreciated that in any of the herein-described assays using shrimp TnC and/or FABP, e.g., an IgE binding assay, an IgE crosslinking assay, or a SPT, the shrimp TnC and/or FABP can be from any source, e.g., chemically synthesized, recombinantly produced, or purified from natural sources.

Diagnostic methods and workflow

In some embodiments, the herein described assays involving shrimp TnC and/or FABP are integrated into a diagnostic protocol that comprises multiple allergy assays, in order to improve the overall diagnostic accuracy for shrimp allergy. For example, the current standard diagnostic approach for shrimp allergy involves a thorough review of clinical history, and is supported by an in vivo skin prick test and a measurement of specific IgE antibodies against a shrimp extract on the ImmunoCAP platform. In addition, double-blind placebo-controlled food challenge (DBPCFC) remains the gold standard to differentiate true allergic and tolerant subjects, but this procedure is labor intensive, expensive and risky to the patients in terms of developing anaphylaxis during the challenge.

In some embodiments, the present disclosure provides methods of detecting a shrimp allergy in a subject, comprising performing two or more allergy assays on the subject, wherein at least one of the assays comprises shrimp TnC and/or shrimp FABP. For example, a method can comprise one or more steps of: performing a thorough review of clinical history, performing a SPT, performing an IgE binding assay, and performing an IgE crosslinking assay such as BAT, wherein at least one of the assays, e.g., the SPT, IgE binding assay, and/or IgE crosslinking assay, comprises shrimp TnC and/or FABP, e.g., recombinant shrimp TnC and/or FABP. Different algorithms involving various combinations of such steps can be evaluated and compared in terms of their diagnostic accuracy, e.g., the specificity and positive predictive value (PPV). Algorithms can be evaluated, for example, by comparing the predicted allergy statuses for test subjects as determined by a given algorithm with the allergy statuses as determined by DBPCFC. Algorithms can also be evaluated in terms of the number of DBPCFCs that need to be conducted to confirm shrimp allergy and tolerance. See, e.g., Examples 5 and 6, below.

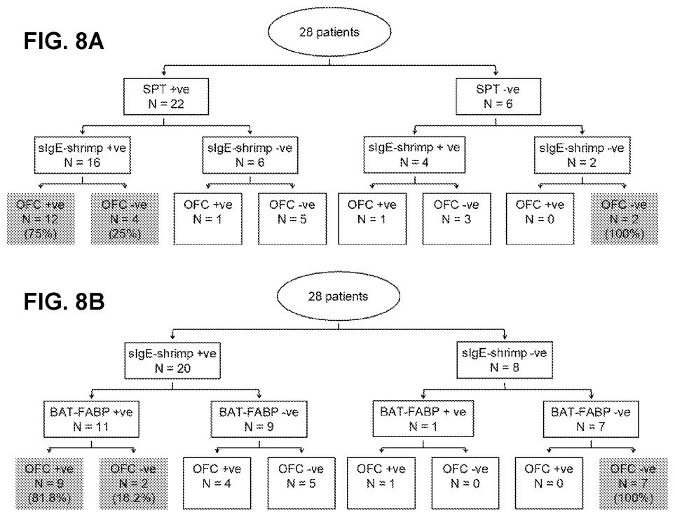

In a particular embodiment, an IgE-crosslinking assay, e.g., BAT-FABP, is used as the second step in a diagnostic algorithm following an IgE binding assay to shrimp extract (FIG. 8B; Example 5). It will be appreciated, however, that variations of this method can be used, e.g., using BAT-TnC instead of (or in addition to) BAT-FABP in the second step, using an IgE binding assay with shrimp TnC and/or FABP instead of a binding test using shrimp extract in the first step, performing the steps in a different order, or including additional assays such as a skin prick test (e.g., using shrimp TnC and/or FABP).

6. Treatment or preventive methods

Upon detection of a likely shrimp allergy in an individual who is being tested using the method described herein, various precautionary measures and, if necessary, therapeutic measurements can be taken to prevent as well as treat shrimp allergy in the person. For instance, the person who has been determined as possibly having a shrimp allergy is be advised to refrain from consuming or even coming into contact with in other fashions (e.g., touching or inhaling) any items, especially food items including additives or supplements, that contain shrimp or shrimp products to prevent the onset of a shrimp allergy. Further, in the event that the person has already come into contact with any shrimp-containing item that might trigger an allergic reaction, whether or not allergic symptoms have already developed, therapeutic intervention may be deployed immediately for treating the allergy by agents such as oral or intravenous steroids and/or anti-histamines. For a case of severe shrimp allergy including anaphylaxis, the use of adrenaline/epinephrine (e.g., injectable epinephrine such as Auvi-Q, EpiPen, Jext, and the like) may be necessary. Moreover, individuals who have been determined as likely to suffer from shrimp allergy may choose to undergo procedures of desensitizing themselves from shrimp allergens and thus overcome shrimp allergy.

7. Kits

The invention also provides kits for the diagnosis of a shrimp allergy in a subject according to the methods of the present invention. The kits typically include a first container that contains a composition comprising or consisting essentially of a shrimp TnC and/or FABP allergen of the invention, and a second container containing a negative control sample that is taken, optionally processed, from a patient who has been confirmed to have no shrimp allergies. Optionally, the kit may include a positive control sample, which is taken (optionally processed) from a subject who has been confirmed to be allergic to shrimp and therefore has a detectable level of IgE specific to shrimp TnC and/or FABP in his or her body. In some embodiments, the kit contains both shrimp TnC and FABP. In some embodiments, the shrimp TnC and/or FABP is recombinant. In some embodiments, the shrimp TnC and/or FABP is from Penaeus monodon. The polypeptide(s) maybe immobilized to a solid substrate, in some cases in the form of an array, and the solid substrate such as an assay plate is suitable for use in an immunoassay such as ELISA or ImmunoCAP.

The kit may further include another container containing a secondary antibody, for example, an anti-IgE (or anti-IgG) antibody, which is optionally conjugated to a detectable label. The kit may also contain one or more reagents, e.g., reagents for performing IgE or IgG binding or crosslinking assays. In addition, the kit may include informational material containing instructions for a user on how to use the kit for performing an assay and determining whether a test subject is likely to suffer from a case of shrimp allergy.

### EXAMPLES

The present invention will be described in greater detail by way of specific examples. The following examples are offered for illustrative purposes only, and are not intended to limit the invention in any manner. Those of skill in the art will readily recognize a variety of noncritical parameters which can be changed or modified to yield essentially the same results.

Example 1. Cloning, expression and purification of recombinant TnC and FABP

Protein sequences of TnC and FABP from Penaeus monodon were downloaded from the Uniprot database (UniProt ID:E7CGC5 and Q1KS35) and reverse translated by MEGA 7.0. The nucleotide sequences coding full-length TnC and FABP were synthesized and cloned into the His-tag expression vector pET30(a)+. His-tagged recombinant TnC and FABP were then expressed in E.coli BL21 (DE3) by IPTG induction and purified using the HisPur cobalt spin columns. Protein concentration and purity were determined by BCA assay and on SDS-PAGE, respectively. IgE reactivity of recombinant TnC and FABP was then confirmed by ELISA using sera from shrimp allergic subjects and non-allergic controls.

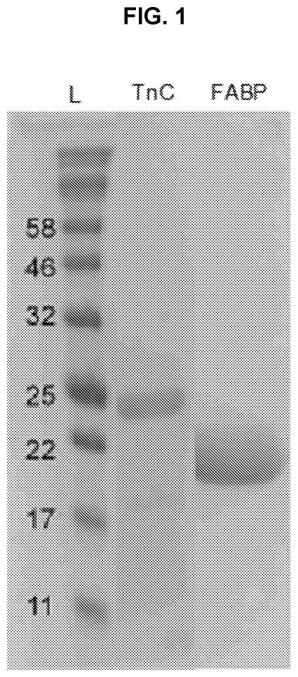

We successfully produced recombinant TnC and FABP using the methodologies described above, and validated their purity of >95% by SDS-PAGE analysis for the subsequent allergy assays (FIG. 1). Briefly, 15 μL of the eluted recombinant protein was mixed with 5 μL of 4X Laemmli sample buffer and loaded into 12% Tris-glycine SDS-PAGE gel. The gel was run at 200V in 1X SDS Running Buffer until the dye front reaches the bottom of the gel. The gel was finally stained with SimpleBlue SafeStain for protein band visualization.

Example 2. IgE-binding assays

Recombinant TnC and FABP can be used as diagnostic markers by incorporating them into IgE-binding assays such as ELISA and ImmunoCAP. For ELISA, 5 μg/ml recombinant TnC or FABP coated onto flat-bottom microtiter plates were blocked with 5% fetal bovine serum (FBS) diluted in PBS at room temperature for 2 h. Serum samples were diluted with blocking buffer at 1:10 and incubated at 4° C. overnight. The plates were then washed with PBST and incubated with biotinylated anti-human IgE antibodies at 1:1000 dilution at room temperature for 1 h. After washing with PBST, the plates were incubated with HRP avidin D at 1:1000 dilution at room temperature for 30 min, washed and incubated with TMB-ELISA substrate solution for color development. The reaction was terminated by adding 2N sulfuric acid to the wells and the optical density (O.D.) at 450 nm was measured using a microplate reader. Results were considered positive if the O.D. is two-fold higher than those of negative controls. Specific IgE levels to recombinant TnC and FABP can also be quantified by ImmunoCAP. The in-house purified recombinant TnC and FABP are covalently bound to ImmunoCAP and the serum specific IgE level can be measured on the Phadia platform using a cut off at 0.35 kUA/L.

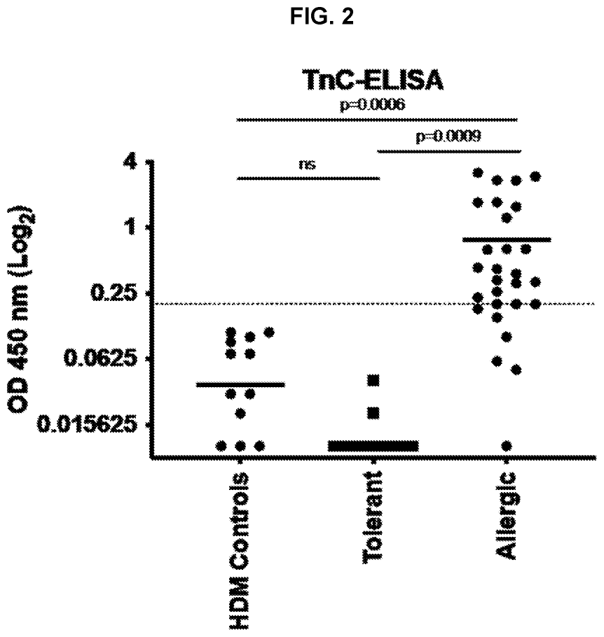

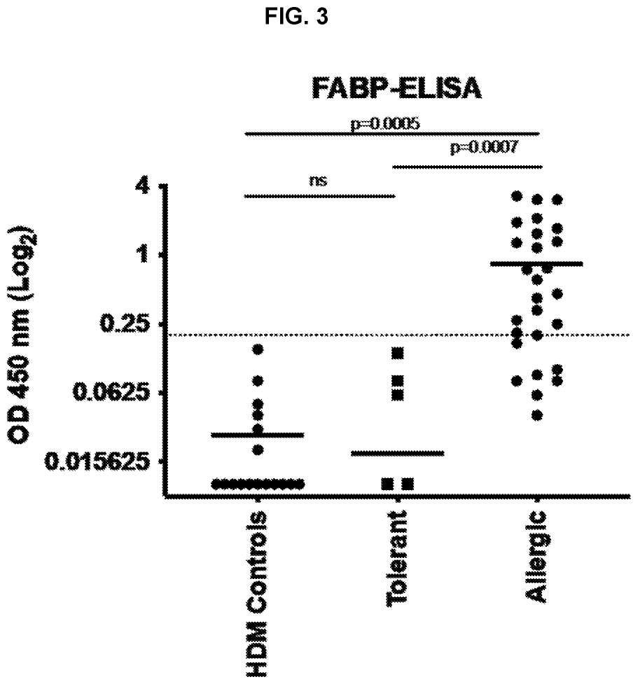

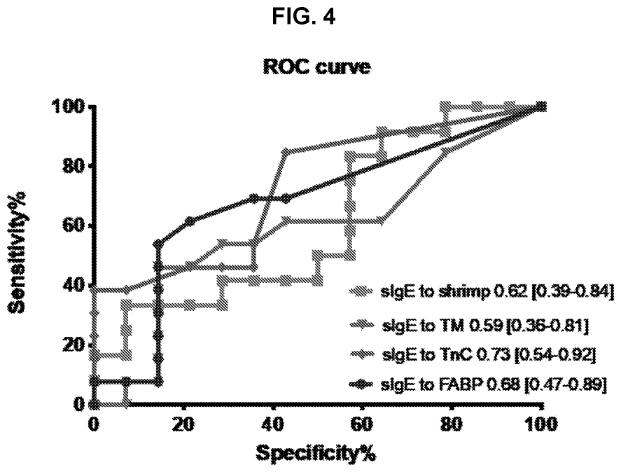

Serum samples were collected from patients who had a positive IgE level to shrimp extract and a reported history of allergic reactions to shrimp (shrimp allergic group; n=30), subjects who had positive sIgE to shrimp extract but without a history of allergic reaction (shrimp tolerant group; n=16) and house dust mite (HDM) controls (n=18) who only had a positive IgE level to dust mite extract but not to shrimp extract. Our ELISA data showed that positive IgE sensitization to TnC and FABP was only found in the shrimp allergic group but not in the tolerant and HDM control groups (FIGS. 2 and 3). On the other hand, 73.3% (22/30) and 66.7% (20/30) allergic subjects were IgE positive to TnC and FABP respectively, suggesting that these two are major shrimp allergens in the Chinese cohort. More importantly, comparing to measuring specific IgE level to shrimp extract and Pen m 1, the “major” shrimp allergen tropomyosin, TnC and FABP represent better diagnostic markers as shown by the higher area under curve (AUC) values in ROC curve analysis (FIG. 4).

Example 3. IgE-crosslinking tests

Recombinant TnC and FABP can be used as diagnostic markers by incorporating them into IgE-crosslinking assays such as the BAT. EDTA-anticoagulated venous blood was collected from shrimp allergic subjects. Each reaction was prepared with 5,000-10,000 ng/mL of allergen (recombinant TnC and FABP described as above), staining reagent (anti-CD63-FITC and anti-CCR3-PE antibody mixture), and EDTA whole blood diluted in stimulation buffer containing IL-3 (2 ng/mL). Positive controls were prepared with anti-FccRT monoclonal antibodies and anti-N-formyl-methionyl-leucyl-phenylalanine (fMLP), respectively, whereas background reactions were assessed with stimulation buffer. The reaction mixtures were then incubated in a 37° C. water bath for 25 minutes. After the addition of lysis buffer to stop the stimulation and centrifugation, stained cells were acquired on a flow cytometer with basophils gated as CCR3^{pos}/SSC^{low}. Upregulation of the basophil marker CD63 was calculated based on the percentage of CD63⁺ cells compared with the total number of identified basophils. In each assay, a minimum of 300 events (i.e., CCR3^{pos} basophils) were recorded. A cut off of 9% CD63⁺ cells was recommended based on our experience.

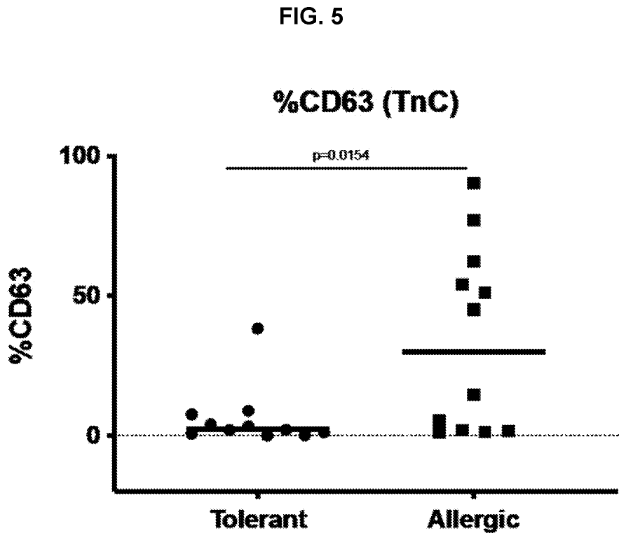

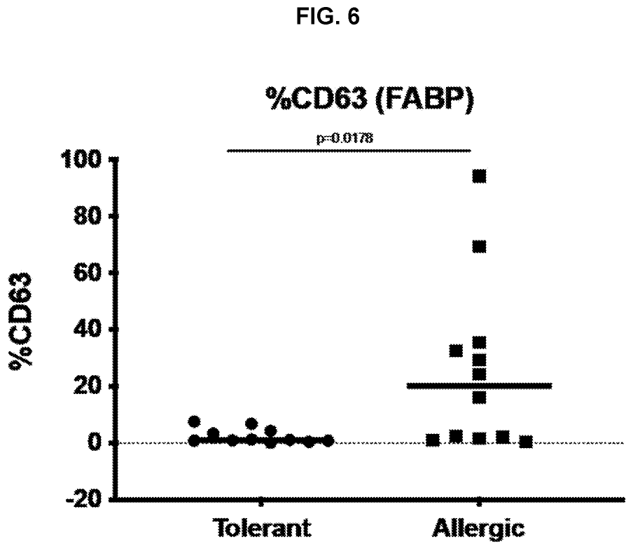

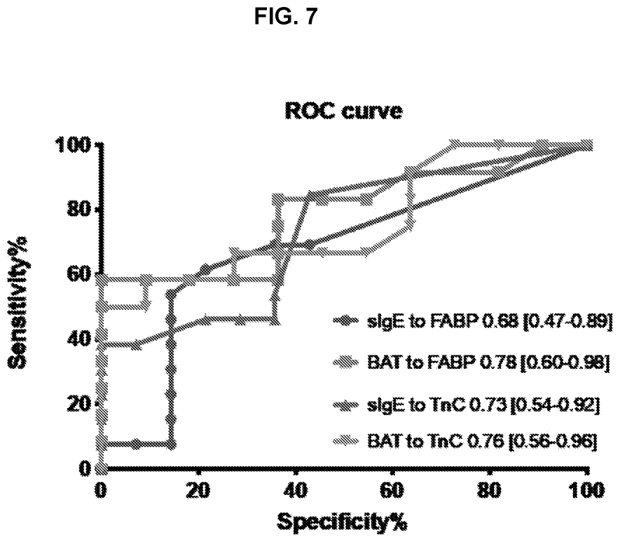

Whole blood was collected from shrimp allergic (n=12) and tolerant subjects (n=14) confirmed by DBPCFC and stimulated against TnC or FABP. It is noted that only basophils from the allergic group showed positive responses upon TnC or FABP stimulation, as indicated by the significantly higher percentage of activated basophil (i.e., % CD63 cells) in patients from the allergic group than those in the tolerant group (FIGS. 5 and 6). When comparing the AUC values among IgE level and IgE crosslinking measurements, it is noted that the use of the IgE crosslinking assay (i.e., BAT) in combination with using FABP and TnC as stimulants has improved diagnostic accuracy (FIG. 7).

Example 4. Skin Prick Test

Recombinant TnC and FABP can be used as diagnostic markers by incorporating them into SPT. These recombinant proteins are dissolved in glycerol/PBS at full-strength. Histamine and normal saline are used as positive and negative controls, respectively. SPT is performed on the volar surface of the forearm of individual patients with single peak lancets and the results are interpreted after 15 min. Reactions with a wheal size of 3 mm is considered positive.

Example 5. Stepwise testing approach incorporating recombinant FABP

The current standard diagnostic approach for shrimp allergy involves a thorough review of clinical history, and supported by in vivo SPT and measurement of specific IgE against shrimp extract on the ImmunoCAP platform. DBPCFC remains the gold standard to differentiate true allergic and tolerant subjects. However, this procedure is labor intensive, expensive and risky to the patients in terms of developing anaphylaxis during the challenge, and thus challenges its clinical implementation.

Examining our pilot data on 28 subjects who had a positive SPT to shellfish (≥3 mm wheal size cut-off) and positive IgE to shrimp (≥0.35 kUA/L) (i.e., conventional stepwise approach), one can see that 12/28 of the DBPCFCs resulted in an adverse food reaction (FIG. 8A). 4/28 subjects fulfilling this criterion indeed passed DBPCFC. This algorithm also successfully identified two shrimp tolerant subjects (100%) that were negative in both SPT and sIgE-shrimp.

The present methods involve the incorporation of the novel recombinant allergens TnC and/or FABP into the existing allergy assays as described herein, and incorporation of the test(s) into the diagnostic algorithm to improve the overall diagnostic accuracy for shrimp allergy. Illustrated with our pilot data that incorporate BAT-FABP as the second step in the diagnostic algorithm after IgE measurement to shrimp extract (FIG. 8B), 10/28 of the DBPCFCs resulted in a reaction when following the criteria of either positive in both sIgE-shrimp and BAT-FABP or positive in only BAT-FABP. Only 2/27 subjects fulfilling these criteria passed DBPCFC. This new algorithm also successfully identified more shrimp tolerant subjects, (i.e., 7 subjects, 100% accuracy) that were negative in both sIgE-shrimp and BAT-FABP.

In terms of comparing the diagnostic accuracy of the two algorithms, the conventional algorithm only presented specificity of 33.3% and positive predictive value (PPV) of 0.75. The present methods (i.e., new algorithm incorporating BAT-FABP into the diagnostic workflow) greatly increased the specificity to 77.8% and the PPV to 0.83. A direct comparison of the diagnostic performance of the two algorithms is indicated in FIG. 8. The new algorithm could also potentially reduce the number of DBPCFC to be conducted to confirm shrimp allergy and tolerance, for example from 14 to 12 challenges comparing the two algorithms.

Example 6. Comparison with existing methods

Shrimp extract and shrimp tropomyosin are the standard biomarkers for shrimp allergy diagnosis, and ImmunoCAP is the most widely adopted platform clinically. Based on our analysis on a total of 28 patients including 14 shrimp allergic subjects and 14 shrimp tolerant subjects confirmed by DBPCFC, measuring IgE to shrimp extract on ImmunoCAP only has a low specificity of 35.0%, whereas that to rPen a 1 yield a specificity of 85.7%. Both of them also showed sub-optimal diagnostic accuracy, as illustrated by area under the curve (AUC) values of 0.62 and 0.59, respectively.

With the successful production of recombinant shrimp TnC and FABP by our laboratory, we showed that measuring IgE to shrimp TnC and FABP by ELISA had a superior diagnostic accuracy, with a specificity of 92.9% and an AUC of 0.73 and 0.68, respectively. On the other hand, we also tested the incorporation of these recombinant allergens into the BAT. Compared to the use of shrimp tropomyosin, which had a sensitivity of 57.1% and an AUC of 0.70, using recombinant TnC and FABP as stimulants in the BAT basophil activation test showed a sensitivity of 64.3% and higher AUC values of 0.78 and 0.76, respectively. Specificity also increased to 92.9% at a cut-off of 9% when using BAT-FABP as diagnostic test comparing to 85.7% of BAT-tropomyosin.

As described above, the incorporation of BAT-FABP as the second step in the diagnostic workflow following IgE measurement to shrimp greatly improves the diagnostic accuracy (i.e., specificity 77.8% and PPV 0.83) compared to the conventional algorithm, which only presented a specificity of 33.3% and a PPV of 0.75. The new algorithm could potentially reduce the number of DBPCFC to be conducted to confirm shrimp allergy and tolerance.

Although the foregoing invention has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference.

1. A method for detecting a shrimp allergy in a subject, the method comprising:
incubating a serum or plasma sample from the subject in the presence of shrimp troponin C (TnC) and/or fatty acid-binding protein (FABP); and
detecting the level of IgE binding to the shrimp TnC and/or FABP in the serum or plasma sample;
wherein a detection of a greater amount of IgE binding in the serum or plasma sample to the shrimp TnC or FABP as compared to the amount in serum or plasma from a non-allergic control indicates that the subject has a shrimp allergy.

2. The method of claim 1, wherein the IgE-binding assay is an ELISA or ImmunoCAP assay.

3-4. (canceled)

5. A method for detecting a shrimp allergy in a subject, the method comprising:
incubating a blood sample from the subject in the presence of shrimp TnC and/or FABP; and
detecting the level of IgE crosslinking in immune cells in the blood sample in the presence of the shrimp TnC and/or FABP;
wherein a detection of a greater amount of IgE crosslinking in immune cells in the blood sample in the presence of the shrimp TnC and/or FABP as compared to the amount in blood from a non-allergic control indicates that that subject has a shrimp allergy.

6. The method of claim 5, wherein the IgE crosslinking assay is a basophil activation test (BAT).

7. The method of claim 6, wherein the BAT is performed using TnC and/or FABP.

8. The method of claim 6, wherein basophil activation is detected by detecting increased numbers of CD63⁺ basophils in the blood sample.

9-10. (canceled)

11. A method for detecting a shrimp allergy in a subject, the method comprising:
pricking the skin of the subject at a location where a sufficient amount of a solution comprising shrimp TnC and/or FABP is applied; and
assessing the reaction of the skin at the location of the skin prick after a specified amount of time;
wherein an allergic reaction of the skin at the site of the skin prick indicates that the subject has a shrimp allergy.

12. The method of claim 11, wherein the specified amount of time is 15 minutes.

13-16. (canceled)

17. A method for detecting a shrimp allergy in a subject, the method comprising:
(i) performing an IgE binding assay on a serum or plasma sample obtained from the subject using one or more shrimp allergens; and
(ii) performing an IgE crosslinking assay on a blood sample obtained from the subject using shrimp troponin C (TnC) and/or fatty acid-binding protein (FABP);
wherein a detection in step (i) of a greater amount of IgE binding to the one or more shrimp allergens in the serum or plasma sample as compared to the amount in serum or plasma from a non-allergic control, and a detection in step (ii) of a greater amount of IgE crosslinking in immune cells in the blood sample in the presence of the shrimp TnC and/or FABP as compared to the amount in blood from a non-allergic control, indicates that that subject has a shrimp allergy.

18. The method of claim 17, wherein the IgE-binding assay is performed using a shrimp extract.

19. (canceled)

20. The method of claim 17, wherein the IgE binding assay is an ELISA or ImmunoCAP assay.

21. The method of claim 17, wherein the IgE crosslinking assay is a basophil activation test (BAT).

22. (canceled)

23. The method of claim 21, wherein basophil activation is detected by detecting increased numbers of CD63⁺ basophils in the blood sample.

24. The method of claim 17, wherein the one or more shrimp antigens, the shrimp TnC, and/or the shrimp FABP is from Penaeus monodon.

25. The method of claim 17, further comprising performing a skin-prick test on the subject using one or more shrimp allergens.

26. The method of claim 25, wherein the skin-prick test is performed using shrimp TnC and/or shrimp FABP.

27. The method of claim 17, wherein the subject is determined to have a shrimp allergy based on steps (i) and (ii), and wherein the method further comprises a step in which the subject is administered a treatment for shrimp allergy.

28. The method of claim 27, wherein the treatment comprises administering an antihistamine or a corticosteroid, or immunotherapy.

29. An expression cassette comprising a polynucleotide encoding Penaeus monodon TnC or FABP or a fragment thereof, operably linked to a heterologous promoter.

30-34. (canceled)

35. A bacterial cell comprising a polynucleotide encoding Penaeus monodon TnC or FABP.

36. (canceled)
